(19) United States
(12) Patent Application Publication
(10) Pub. No.: US 2020/0126648 A1
(43) Pub. Date: Apr. 23, 2020

(54) HOLISTIC PATIENT RADIOLOGY VIEWER

(71) Applicant: KONINKLIJKE PHILIPS N.V., EINDHOVEN (NL)

(72) Inventors: Nicole SCHADEWALDT, NORDERSTEDT (DE); Amir Mohammad TAHMASEBI MARAGHOOSH, ARLINGTON, MA (US); Lyubomir Georgiev ZAGORCHEV, BIRLINGTON, MA (US); Sandeep Madhukar DALAL, WINCHESTER, MA (US); Daniel BYSTROV, HAMBURG (DE); Astrid Ruth FRANZ, HAMBURG (DE)

(21) Appl. No.: 16/604,317

(22) PCT Filed: Apr. 13, 2018
(86) PCT No.: PCT/EP2018/059491

§ 371 Date: Oct. 10, 2019

Related U.S. Application Data
(60) Provisional application No. 62/486,480, filed on Apr. 18, 2017.

(51) Int. Cl. G16H 15/00; G16H 50/70; G16H 30/20; G16H 80/00; G16H 10/60

(52) U.S. Cl. CPC G16H 15/00; G16H 50/70; G16H 10/60; G16H 80/00; G16H 30/20

(57) ABSTRACT

A radiology viewer includes at least one electronic processor (10, 20), at least one display (12), and at least one user input device (14, 16, 18). The display shows at least a portion of a radiology image (30) in an image window (40), and at least a portion of a radiology report (32) in a report window (42). A selection is received of an anatomical feature shown in the image window, and a corresponding passage of the radiology report is identified and highlighted in the report window. A selection is received of a passage of the radiology report shown in the report window, and a corresponding anatomical feature of the at least one radiology image is identified and highlighted in the image window. The highlighting operations use image anatomical feature tags and report clinical concept tags generated using a medical ontology (56) and an anatomical atlas (62).

### FIELD

The following relates generally to the medical imaging arts, medical image viewer and display arts, and related arts.

### BACKGROUND

In the modern medical practice, the patient is expected to be an active participant in his or her medical care. For example, the patient must provide informed consent to various medical procedures, if physically and mentally competent to do so. To this end, it is important that the patient understand the findings of medical examinations such as radiology examinations.

However, most lay patients (that is, patients without medical training) are unfamiliar with detailed anatomy, much less the visualization of such anatomy as presented in medical images. In a typical radiology workflow, the images of a radiology examination are interpreted by a skilled radiologist who prepares a radiology report summarizing the radiologist's clinical findings. However, the radiology report uses advanced clinical language and anatomical and clinical terminology that is generally unfamiliar to the lay patient. The usual approach for conveying the substance of the radiology examination results to the patient is by way of the patient's physician or a medical specialist explaining these results to the patient in a “one-on-one” consultation. However, this is time consuming for the medical professional, and moreover not all medical professionals are proficient at explaining complex medical findings in a way that is readily understood by the lay patient.

The following discloses certain improvements.

### SUMMARY

In one disclosed aspect, a radiology viewer comprises an electronic processor, at least one display, at least one user input device, and a non-transitory storage medium storing: instructions readable and executable by the electronic processor to retrieve a radiology examination including at least one radiology image and a radiology report from a radiology examinations data storage; instructions readable and executable by the electronic processor to retrieve or generate a set of image tags identifying anatomical features in the at least one radiology image and a set of report tags identifying clinical concepts in passages of the radiology report; instructions readable and executable by the electronic processor to display at least a portion of the at least one radiology image in an image window shown on the at least one display and to display at least a portion of the radiology report in a report window shown on the at least one display; instructions readable and executable by the electronic processor to receive via the at least one user input device a selection of an anatomical feature shown in the image window and to identify at least one related passage of the radiology report using the set of image tags, the set of report tags, and an electronic medical ontology and to highlight the at least one related passage of the radiology report in the report window; and instructions readable and executable by the electronic processor to receive via the at least one user input device a selection of a passage of the radiology report shown in the report window and to identify at least one related anatomical feature of the at least one radiology image using the set of image tags, the set of report tags, and the electronic medical ontology and to highlight the at least one related anatomical feature of the at least one radiology image in the image window.

In another disclosed aspect, a non-transitory storage medium stores instructions readable and executable by an electronic processor operatively connected with at least one display, at least one user input device, and a radiology examinations data storage to perform a radiology viewing method operating on at least one radiology image and a radiology report. In the radiology viewing method, at least a portion of the at least one radiology image is displayed in an image window shown on the at least one display. At least a portion of the radiology report is displayed in a report window shown on the at least one display. Using a set of image tags identifying anatomical features in the at least one radiology image, a set of report tags identifying clinical concepts in passages of the radiology report, and an electronic medical ontology, at least one of the following is performed: (1) receiving via the at least one user input device a selection of an anatomical feature shown in the image window, identifying at least one related passage of the radiology report, and highlighting the at least one related passage of the radiology report in the report window; and (2) receiving via the at least one user input device a selection of a passage of the radiology report shown in the report window, identifying at least one related anatomical feature of the at least one radiology image, and highlighting the at least one related anatomical feature of the at least one radiology image in the image window.

In another disclosed aspect, a radiology viewer includes at least one electronic processor, at least one display, and at least one user input device. The display shows at least a portion of a radiology image in an image window, and at least a portion of a radiology report in a report window. A selection is received of an anatomical feature shown in the image window, and a corresponding passage of the radiology report is identified and highlighted in the report window. A selection is received of a passage of the radiology report shown in the report window, and a corresponding anatomical feature of the at least one radiology image is identified and highlighted in the image window. The highlighting operations use image anatomical feature tags and report clinical concept tags generated using a medical ontology and an anatomical atlas.

One advantage resides in providing a radiology viewer that provides intuitive visual linkage between radiology report contents and related features of the radiology images which are the subject of the radiology report.

Another advantage resides in providing a radiology viewer that facilitates understanding of a radiology examination by a lay patient.

Another advantage resides in providing a radiology viewer that presents radiology findings with visual representation of the anatomical context.

Another advantage resides in providing a radiology viewer that graphically links clinical concepts presented in the radiology report with anatomical features represented in the underlying medical images.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a radiology viewer as disclosed herein.

FIGS. 2-4 diagrammatically illustrate processing performed by the radiology viewer of FIG. 1.

FIG. 5 diagrammatically illustrates screenshots of the radiology viewer of FIG. 1 for three successive radiology examinations of a patient, including visual renderings of linkages between selected clinical concepts of the radiology reports and related anatomical features of the underlying medical images.

FIG. 6 diagrammatically illustrates a screenshot of the radiology viewer of FIG. 1 showing user interaction to explore the general anatomy.

### DETAILED DESCRIPTION

Disclosed herein are radiology viewers that determine linkages between clinical concepts presented in the radiology report of a radiology examination and related anatomical features in the underlying medical images, and that graphically present these linkages to the patient or other user in an intuitive fashion. The disclosed improvements are premised in part on the recognition that understanding of the results of a radiology examination generally requires synthesis of contents of the radiology report with features shown in the underlying medical images.

In the case of the user being a lay patient or other layperson, it is further recognized that the user may in general be unfamiliar with the anatomical context of clinical findings of the radiology report—accordingly, the disclosed radiology viewer provides for the user to identify features in the images by selecting the feature at which point a contextual explanation of the selected feature is presented, and any associated content of the radiology report is highlighted. Conversely, by selecting a passage of the radiology report the related feature(s) of the underlying medical image(s) are highlighted and identified by their anatomical terms (e.g. “kidney”, “lymph node”, “left thalamus”, et cetera).

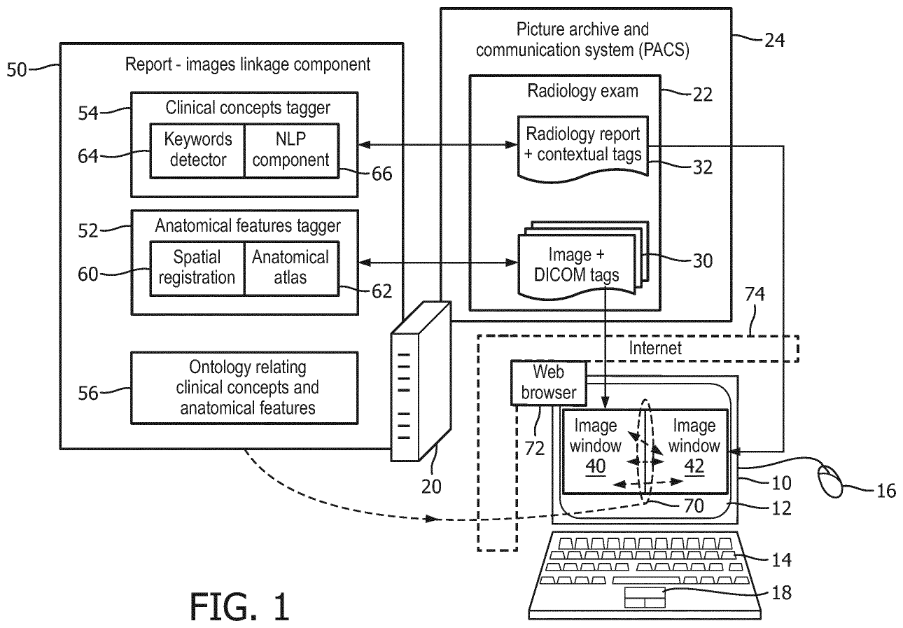

With reference to FIG. 1 an illustrative radiology viewer comprises a viewer workstation 10 including or operatively connected with at least one display 12 (e.g. an LCD display, plasma display, or so forth) and at least one user input device, such as an illustrative keyboard 14, mouse 16, trackpad 18, touch-sensitive overlay of the display 12, and/or so forth. The illustrative viewer workstation 10 is embodied as a desktop or notebook computer, but alternatively could be embodied as a tablet computer, smartphone, or other mobile device. The illustrative radiology viewer also includes or is in operative connection with a server computer 20. As is known in the computing arts, the viewer workstation 10 includes an electronic processor (e.g. a microprocessor) and the server computer 20 includes an electronic processor (e.g. a microprocessor, or the server computer 20 may comprise a computing cluster, cloud computing resource, or the like that includes a plurality of electronic processors). Moreover, it is contemplated in some embodiments for all disclosed processing to be performed by the electronic processor of the viewer workstation 10, in which case the server computer 20 may optionally be omitted.

The radiology viewer workstation 10 retrieves a radiology examination 22 from a radiology examinations data storage, such as an illustrative Picture Archiving and Communication System (PACS) 24. Diagrammatic FIG. 1 illustrates a single illustrative radiology examination 22; however, it will be understood that that PACS 24 typically stores all radiology reports for a given patient, and for all patients who have been imaged by the radiology department or other radiology imaging service, suitably indexes by parameters such as patient identifier (PID), date of examination, date of radiology reading, imaging modality, imaged anatomical region, and/or so forth. The illustrative radiology examination 22 includes a set of radiology images 30 and a radiology report 32. The radiology images 30 could be as few as a single image, though in most cases the radiology examination 22 will, as shown in FIG. 1, include a plurality of images. Each image typically has metadata stored with the images, for example as image tags in a standard DICOM format. These tags may, for example, identify PID, date of acquisition, imaging acquisition parameters, and so forth. The radiology images 30 may in general be acquired using any suitable imaging modality, such as transmission computed tomography (CT), magnetic resonance (MR) imaging, positron emission tomography (PET) imaging, single photon emission computed tomography (SPECT) imaging, or so forth. The radiology images 30 may be a stack of two-dimensional (2D) image slices, e.g. axial image slices, collectively forming a three-dimensional (3D) image, or may be acquired directly as a 3D image. As is known in the art, the images 30 may optionally have been acquired with contrast enhanced by way of an exogenous contrast agent administered to the patient prior to imaging data acquisition. In the case of nuclear medicine imaging (e.g. PET or SPECT), the images 30 are acquired after administration of a suitable radiopharmaceutical to the patient, typically with some intake delay imposed between the radiopharmaceutical administration and the imaging data acquisition to allow the radiopharmaceutical to be taken up by the target tumor or organ.

The radiology report 32 is a report prepared by a radiologist or other medical professional which presents a summary of observations on the images 30 and clinical findings determined by the radiologist via review of the radiology images 30. The radiology report 32 may also be prepared based on other information available to the radiologist, such as the patient's medical history, and/or comparison of the radiology images 30 of the current radiology examination 22 with past radiology examinations of the patient (not shown in FIG. 1), and/or so forth. The author of the radiology report 32 is generally a radiologist or other trained medical professional and is written to convey medical findings to other trained medical professionals such as the patient's general-practice doctor, an oncologist, or the like. Accordingly, the radiology report 32 is generally written using domain-specific medical and anatomical terminology that is often unfamiliar to the lay patient. The radiology report 32 is a text-based report, meaning that the report 32 consists mostly or entirely of text; however in some embodiments the text-based report 32 may include some non-text content such as embedded “thumbnail” representations of one or more of the radiology images 30.

The radiology viewer workstation 10 retrieves the radiology examination 22 from the PACS 24, including the radiology image(s) 30 and the radiology report 32. The viewer workstation 10 presents these data in two windows: an image window 40 in which at least a portion of the at least one radiology image 30 is displayed; and a report window 42 in which at least a portion of the radiology report 32 is displayed. In some embodiments, the image window 40 provides various image manipulation functions operable by the user via the at least one user input device 14, 16, 18—for example, the image manipulations may include zoom-in and zoom-out operations, a pan operation, and so forth. Depending upon the zoom magnitude, only a portion of an image may be seen in the image window 40. Likewise, depending upon the length of the radiology report 32, only a portion of that report 32 may be shown at any given time in the report window 42, and the user is provided with various control functions such as scroll operations, text font size adjustments, and/or so forth operable by the user via the at least one user input device 14, 16, 18. As shown in FIG. 1, the illustrative viewer workstation 10 shows the image window 40 and the report window 42 simultaneously, in a side-by-side arrangement in the illustrative example. However, it is contemplated to employ other approaches, such as displaying only one of these windows at any given time and providing a hotkey combination such as <ALT>-<TAB> to switch between which window is currently displayed. In another contemplated variant the windows 40, 42 may be configurable in a partially overlapping arrangement. Moreover, while in FIG. 1 a single display 12 is shown which displays both windows 40, 42, the viewer workstation may include two (or more) displays in other embodiments, e.g. two different physical monitors, and each window may then be displayed on its own display.

In improved radiology viewer embodiments disclosed herein, linkages are determined between clinical concepts presented in the radiology report 32 of the radiology examination 22 and related anatomical features in the underlying medical images 30 of the radiology examination 22, and the radiology viewer graphically presents these linkages to the patient or other user in an intuitive fashion. This promotes synthesis of contents of the radiology report with features shown in the underlying medical images. While such assistance may be of value to a radiologist, this assistance is of particular value for lay patient consumption of the radiology examination 22, as the lay patient is generally unfamiliar with clinical terminology, anatomical terminology, and the ways in which various imaging modalities capture anatomical features.

To provide these features, a report-images linkage component 50 is provided. The illustrative linkage component 50 is implemented on the server computer 20, which may be the same server computer 20 that implements the PACS 24 (as shown) or may be a different computer server in communication with the PACS. The linkage component includes an anatomical features tagger 52 for generating a set of image tags identifying anatomical features in the at least one radiology image 30, a clinical concepts tagger 54 for generating a set of report tags identifying clinical concepts in passages of the radiology report 32, and a medical ontology 56 for linking the clinical concepts and the anatomical features.

The illustrative anatomical features tagger 52 includes a spatial registration component 60 which spatially aligns (i.e. registers) the image(s) 30 with an anatomical atlas 62, and generates the set of image tags by associating image features of the anatomical atlas 62 with corresponding spatial regions of the spatially registered at least one radiology image. It is to be understood that the anatomical atlas 62 is typically not a single representation of a human, but rather is a three-dimensional reference space with multi-dimensional annotations of positions and properties of multiple objects, which may be overlapping and/or mutually exclusive. For example, the anatomical atlas 62 may represent both male and female organs simultaneously (with only one gender typically matching with a given image 30). Besides organs, the anatomical atlas 62 may optionally also identify reference points (e.g. the top of the lungs) or regions (e.g. abdominal region) or any other anatomical objects which can be spatially specified. The anatomical atlas may also encode non-spatial characteristics of an anatomical object, e.g. typical CT-level-window-settings for that object or typical appearances in standard MR sequences or any other type of characteristics relevant for identifying or evaluating this object in a radiologic image. Thus, the term “anatomical atlas” here means a reference space encoding multiple types of information on the human body. The resulting tags may be stored in a suitable storage space—in the illustrative example, the image tags are stored as metadata with the image(s) 30 as DICOM tags, which conveniently leverages the existing DICOM tagging framework; however, other tag storage formalisms are contemplated. It is also contemplated to employ manual tagging, e.g. to identify patient-specific anatomical features that may not be included in the atlas 62, such as tumors.

The illustrative clinical concepts tagger 54 employs a keywords detector 64 to identify keywords in the radiology report 32 corresponding with entries of the medical ontology 56, and the set of report tags is generated by associating passages of the radiology report 32 containing the identified keywords with clinical concepts described in the corresponding entries of the medical ontology 56. In another approach, a natural language processing (NLP) component 66 performs natural language processing on the radiology report 32 to identify passages of the radiology report corresponding with entries of the medical ontology, and the set of report tags is generated by associating the identified passages of the radiology report with clinical concepts described in the corresponding entries of the medical ontology. However generated, the resulting report tags are stored in a suitable storage space—in the illustrative example, the report tags are stored as metadata associated with the radiology report 32; however, other tag storage formalisms are contemplated.

The radiology viewer leverages the thusly generated image tags and report tags to enable the display of automated linkages 70 between user-selected anatomical features of the images 30 and corresponding passages of the radiology report 32; or, conversely, enables automated display of linkages 70 between user-selected passages of the radiology report 32 and corresponding anatomical features of the images 30. For example, if the user selects the liver in a radiology image then the image tags are consulted to determine that the point selected in the image is the liver, then the report tags are searched to identify clinical concepts (if any) relating to the liver by searching those clinical concepts in the ontology 56 to detect references of the concepts to the liver, and finally the corresponding passages of the radiology report 32 are highlighted in the report window 42. Conversely, if the user selects a passage containing the keyword “cirrhosis” in the radiology report 32, then the report tags are consulted to determine that the selected passage pertains to the clinical concept of cirrhosis of the liver, then the image tags are searched to identify the liver in the radiology image(s) 30, and finally the identified liver anatomical feature is highlighted in the image window 40.

In displaying the linkages 70, highlighting of a selected anatomical feature and corresponding report passage(s), or conversely highlighting of a selected report passage and corresponding anatomical feature(s), may employ highlighting. The term “highlight” as used herein is intended to denote any display feature used to emphasize the highlighted image feature in a (portion of) a radiology image displayed in the image window 40, or to denote any display feature used to emphasize the highlighted passage of a (portion of) a radiology report displayed in the report window 42. The highlighting of an image feature may comprise, for example, highlighting an image feature by coloring it with a designated color, highlighting an image feature by superimposing a boundary contour (optionally having a distinctive color) delineating the boundary of the image feature, or so forth. The highlighting of a report passage may comprise, for example, employing a highlighting text background color, a highlighting text color, a text feature such as underscore, flashing text, or the like, or so forth. In some embodiments, both the user-selected image feature or report passage and the identified related passage or image feature are highlighted using the same highlighting, such as employing the same color or pattern for highlighting both the image feature and the report passage. Where the image window 40 and the report window 42 are shown simultaneously, e.g. side-by-side as in illustrative FIG. 1, it is also contemplated to depict the linkages 70 using connecting arrows connecting the image feature(s) in the image window 40 and the corresponding report passage(s) in the report window 42, as diagrammatically indicated by the connecting double-headed arrows shown in FIG. 1.

As previously mentioned, while the illustrative embodiment implements the report-images linkage component 50 on the (same) server computer 20 that implements the PACS 24, other configurations are contemplated. For example, the linkages component 50 and PACS 24 may be implemented on different server computers, or in another embodiment the linkages component 50 may be implemented on the viewer workstation 10. In the illustrative embodiment, viewer functions such as constructing and displaying the windows 40, 42 and receiving user inputs via the user input device(s) 14, 16, 18 are implemented on the electronic processor of the viewer workstation 10, while more computationally complex linkages creation 50 is performed on the server computer 20 which generally has greater computing power. In the illustrative example of FIG. 1, the viewer functions are implemented in the form of a web application or web page run by a web browser 72, and the PACS 24 and linkage component 50 (or, more generally, the server computer 20) is accessed via the Internet 74. It will also be appreciated that the disclosed radiology viewer functions may be embodied by a non-transitory storage medium, such as a hard disk drive or other magnetic storage medium, an optical disk or other optical storage medium, a solid state drive (SSD), FLASH memory, or other electronic storage medium, various combinations thereof, or so forth. Such non-transitory storage medium stores instructions readable and executable by an electronic processor (e.g. of the viewer workstation 10 and/or the server computer 20) to perform the disclosed viewer functions.

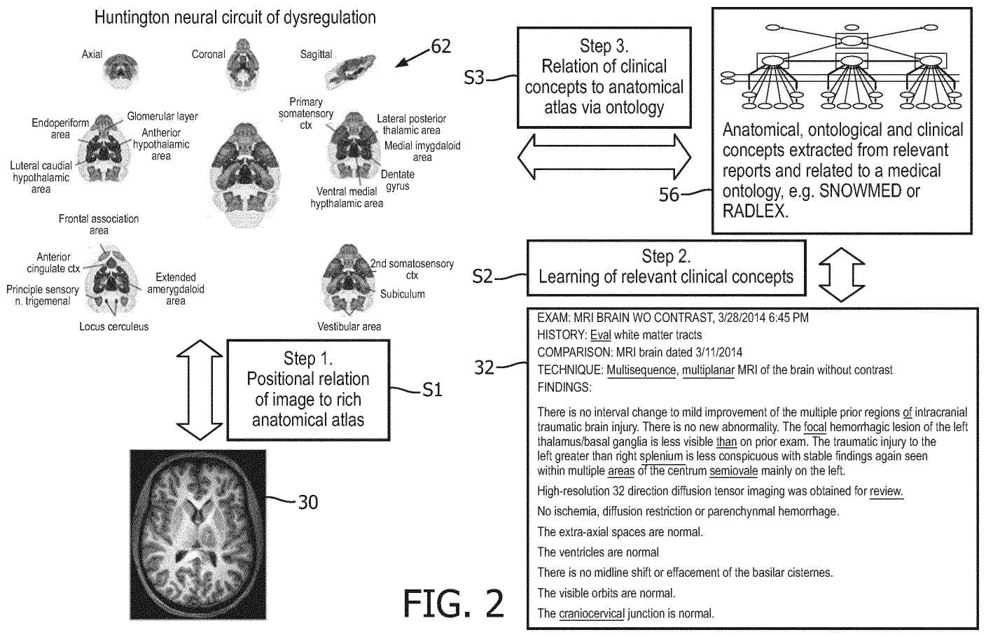

With reference to FIG. 2, the processing performed by the report-images linkage component 50 of FIG. 1 is shown in diagrammatic representation. A radiology image 30 is processed in a step S1 by the anatomical features tagger 56, with reference to the anatomical atlas 62, to generate the anatomical feature tags labeling anatomical features of the image 30. Step S1 entails registration of the medical image 30 to a reference space. Some suitable approaches for this registration are described, by way of non-limiting illustration, in: Pauly et al., “Fast Multiple Organs Detection and Localization in Whole-Body MR Dixon Sequences”, in MICCAI 2011 (14^{th} Int'l Conf. on Medical Image Computing and Computer Assisted Intervention, September 2011); Criminisi et al., “Regression Forests for Efficient Anatomy Detection and Localization in Computed Tomography Scans”, in Medical Image Analysis (MedIA), Elsevier, 2013. The tagging of the anatomical features may include delineating their spatial extent by reference to the atlas 62, and optionally also be using automated contouring starting with the base contour provided by the atlas 62, e.g. using a contour curve or surface that is iteratively deformed to match edges of the anatomical feature.

In parallel, the radiology report 32 is processed in a step S2 by the clinical concepts tagger 54, with reference to the medical ontology 56, to generate the clinical concepts tags labeling passages of the radiology report 32 as to the contained clinical concepts. This may entail keyword detection using the keywords detector 64, and/or more sophisticated processing performed by the natural language processing (NLP)-based engine or component 66, to extract findings or other clinical concepts in the radiology report 32. Keywords in the radiology report 32 are identified with entries of the medical ontology 56, and the set of report tags is generated by associating passages of the radiology report 32 containing the identified keywords with clinical concepts described in the corresponding entries of the medical ontology 56. Additionally or alternatively, natural language processing is performed on the radiology report 32 to identify passages of the radiology report 32 corresponding with entries of the medical ontology 56, and the set of report tags is generated by associating the identified passages of the radiology report 32 with clinical concepts described in the corresponding entries of the medical ontology 56. Both approaches can be combined. In one non-limiting approach, the radiology report 32 is first analyzed by the NLP engine 66 to determine sections, paragraphs, and sentences, and to determine and extract the specific body part and/or organ references from the delineated sentences. The referenced medical ontology 56 may, for example, be a standard medical ontology such as RADLEX or SNOMED CT. The clinical concepts (e.g. findings such as abnormalities, disorders, and/or so forth) are extracted and suitable contextual tags are generated labeling the report passages with the contained clinical concepts.

The steps S1 and S2 may be performed as pre-processing, e.g. at the time the radiology report 32 is filed by the radiologist. Thereafter, the generated anatomical feature tags may be stored as DICOM tags with the images 30, and the generated clinical concept tags are suitably stored with the radiology report 32. Thereafter, when the patient or other user views the radiology examination 22 using the radiology viewer workstation 10, in a step S3 when the user selects an image location or a report passage, the anatomy corresponding to the image location or the clinical concept contained in the passage are determined by referencing the image tags or report tags, respectively, and the ontology 56 is referenced to identify the corresponding report passage(s) or image anatomical feature(s). Thus, via the common ontology 56, clinical concepts and anatomical features are linked. In some embodiments, the linkage step S3 is extended over multiple radiology examinations to identify relations of different time-points in the different examinations. In this way, due to the link with the images, a patient can follow the genesis and/or evolution of an anatomical feature over multiple time points represented by different radiology examinations, even if the structure is not remarked upon in one or more of the radiology reports. For example, if a tumor appears in the kidney, the patient may look at the changes in the kidney across successive radiology examinations, without having to know how to find the kidney in the images of each examination, via the anatomical feature tags.

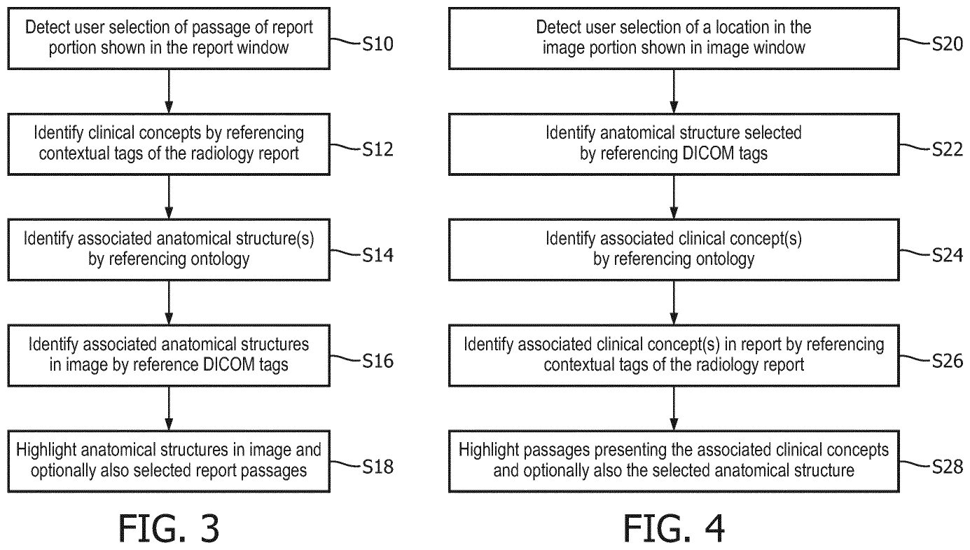

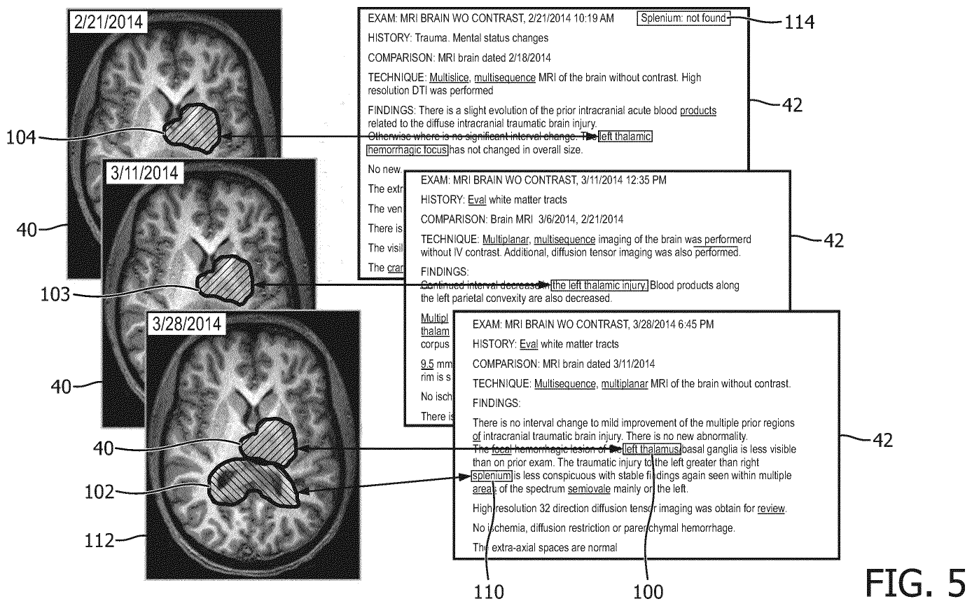

With reference to FIGS. 3 and 4, illustrative processing for executing the step S3 of FIG. 2 are described. FIG. 3 depicts the process for highlighting relevant anatomical feature in the image in response to user selection of a passage of the radiology report. In an operation S10, the user selection of the report passage at the workstation 10 using one of the user interface devices 14, 16, 18 is detected. For example, the user may click on a word or sentence of the report. In an operation S12, the clinical concepts described or mentioned in the selected passage are identified by referencing the contextual tags of the radiology report 32. In an operation S14, the ontology 56 is consulted to identify corresponding anatomical feature(s) that are related to the identified clinical concept. In an operation S16, the image tags are consulted to identify the corresponding anatomical feature(s) in the radiology image. In an operation S18, the anatomical feature(s) are highlighted in the image (portion) displayed in the image window 40, and optionally the selected passage of the report is also highlighted in the report window 42.

FIG. 4 depicts the process for highlighting relevant clinical concept(s) in the radiology report in response to user selection of a location in the image. In an operation S20, the user selection of the location in the image at the workstation 10 using one of the user interface devices 14, 16, 18 is detected. For example, the user may click on a location in the image (portion) shown in the image window 40. Other user selection approaches may be employed, e.g. the patient may select an image region by selecting a rectangular, circular or other shaped region, or may draw a line and ask for the object below the line. More generally, in the operation S20 the user selects a region of the image (e.g. a point, line, area, volume). In an operation S22, the anatomical feature at the selected location is identified by referencing the image anatomical feature tags stored in the DICOM annotations of the displayed radiology image 30. In an operation S24, the ontology 56 is consulted to identify corresponding clinical concept(s) that are related to the identified anatomical feature. In an operation S26, the contextual tags of the radiology report 32 are consulted to identify the corresponding passage(s) in the radiology report 32 that describe or mention the associated clinical concept(s). In an operation S28, the corresponding report passage(s) are highlighted in the report (portion) displayed in the report window 42, and optionally the selected anatomical feature is also highlighted in the image (portion) shown in the image window 40.

It should be noted that in the step S1 of FIG. 2, the image anatomical feature tags are generated automatically using the anatomical atlas 62. Thus, these anatomical feature tags are not reliant upon the accuracy of any image tagging performed by the radiologist during the reading of the radiology examination. In particular, while DICOM tags may be generated to record the radiologist's labeling of image features, these radiologist-generated DICOM tags are not relied upon for operation of the radiology viewer. Rather, the anatomical feature tags automatically generated in step S1 by the anatomical features tagger 52 of FIG. 1 are the tags used by the viewer. These automatically generated anatomical feature tags may optionally be stored as DICOM tags for convenience.

The radiology report viewer can optionally operate to provide viewing of three-dimensional (3D) imaging datasets. For example, the patient or other user can be offered browsing functionality to “flip through” slices of a 3D image. In this regard, it may also be noted that in some instances the image slice currently shown in the image window 40 when a passage of the report 42 is selected may not show the corresponding image feature (or may not optimally show that feature). In such case, the image window 40 may be updated to present the appropriate image slice, either automatically (in some embodiments) or after querying the user as to whether the user wishes to switch to the optimal image slice for depicting the selected report passage (in other embodiments).

The medical ontology 56 employed for mining clinical concepts is generally a domain-specific ontology, specifically a highly specialized medical or radiology ontology. However, for assisting the lay patient in understanding his or her radiology examination, it is contemplated to augment the domain-specific ontology content with lay terms that may be more comprehensible to the patient. For example, terms such as “cardiac” may be augmented by “heart”, or so forth.

In some contemplated embodiments, the steps S1 and S2 of FIG. 2 are performed “offline”, i.e. at the time of creation of the radiology report 32, and the generated anatomical feature and clinical concept tags are stored, e.g. with the images 30 and report 32 respectively as shown in FIG. 1. The step S3 is then performed in real-time as the user selects an image location or report passage, and step S3 then identifies and highlights corresponding report passage(s) or image anatomical feature(s). The processing executing the step S3 may, in some embodiments, be performed locally at the viewer workstation 10, e.g. as a browser plug-in, a program running on a desktop or notebook computer, a cellphone or tablet computer app, or so forth. In these embodiments, a copy of the medical ontology 56 (or at least relevant portions thereof) is suitably stored on the viewer workstation 10. Alternatively, the step S3 could be performed at the server 20 and the results downloaded to the viewer workstation 10 via the Internet 74.

In the following, some illustrative examples are presented.

With continuing reference to FIG. 1 and further reference to FIG. 5, an example is shown of the radiology viewer display including the image window 40 and report window 42 for three consecutive brain exams dated: Feb. 21, 2014 (top display example); Mar. 11, 2014 (middle display example); and Mar. 28, 2014 (bottom display example). The patient can select structures in the text of the report (or report portion) shown in the report window 42, and corresponding anatomical feature(s) are identified in the matching image as per the process of FIG. 3. The identification can extend to prior examination reports using the anatomical feature tags of those prior images. In the illustrative example of FIG. 5, the selected passage 100 in the report window 42 for the latest examination dated Mar. 11, 2014 contains the clinical concept of “left thalamus”, and the corresponding anatomical feature 102 (i.e. the left thalamus) is highlighted in the image window 30. The left thalamus anatomical feature is also highlighted by highlighting 103, 104 in the earlier examinations (which may be displayed in separate windows, for example), optionally along with mentions of the corresponding clinical feature in the earlier examinations. As another example, the user has similarly, the user highlighted a passage 110 containing the clinical concept of “splenium” and the corresponding anatomical feature 112 (the splenium structure) is highlighted. The splenium is not found in the oldest report, which is remarked upon on top of the page in a notation 114. More generally, the absence of the corresponding anatomical feature (or absence of a corresponding passage in the case of a highlighted anatomical feature) is identified.

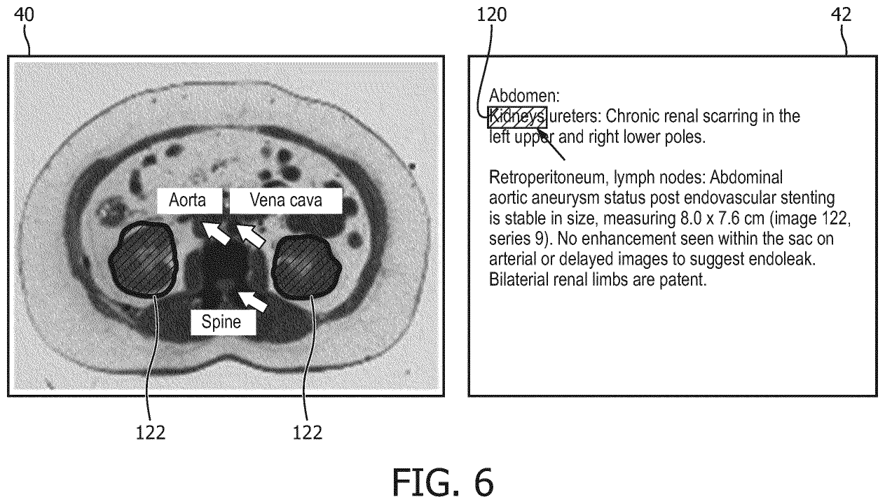

With reference to FIG. 6 another example is shown, in this case an abdominal image shown in the image window 40 and corresponding report shown in the report window 42. In this example, the patient explores additional structures in the image to better understand the anatomy. Here, mouse-over explanations to three different mouse pointer positions (selected anatomical features) are depicted: Aorta, Vena Cava and Spine. That is, as the user moves the mouse over the aorta region, the label “Aorta” pops up. Similarly, as the user moves the mouse over the vena cava region, the label “Vena Cava” pops up. Similarly, as the user moves the mouse over the spinal region, the label “Spine” pops up. These labels may appear briefly and disappear when the mouse is moved out of the region, or alternatively may persist until the user takes some action to remove the label (e.g. clicking on an “X” at a corner of the label, not shown in FIG. 6). As another illustrative example, as the user selects a text passage 120 of the report portion in the report window 42 containing the clinical concept “kidneys”, the corresponding anatomical features (the kidneys) are emphasized by highlighting 122 in the corresponding image in the image window 40.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A radiology viewer comprising:
an electronic processor;
at least one display;
at least one user input device; and
a non-transitory storage medium storing:
instructions readable and executable by the electronic processor to retrieve a radiology examination including at least one radiology image and a radiology report from a radiology examinations data storage,
instructions readable and executable by the electronic processor to retrieve or generate a set of image tags identifying anatomical features in the at least one radiology image and a set of report tags identifying clinical concepts in passages of the radiology report;
instructions readable and executable by the electronic processor to display at least a portion of the at least one radiology image in an image window shown on the at least one display and to display at least a portion of the radiology report in a report window shown on the at least one display;
instructions readable and executable by the electronic processor to receive via the at least one user input device a selection of an anatomical feature shown in the image window and to identify at least one related passage of the radiology report using the set of image tags, the set of report tags, and an electronic medical ontology and to highlight the at least one related passage of the radiology report in the report window and to further highlight the selected anatomical feature using a same emphasis as the highlighting of the related passage of the radiology report; and
instructions readable and executable by the electronic processor to receive via the at least one user input device a selection of a passage of the radiology report shown in the report window and to identify at least one related anatomical feature of the at least one radiology image using the set of image tags, the set of report tags, and the electronic medical ontology and to highlight the at least one related anatomical feature of the at least one radiology image in the image window and to further highlight the selected passage of the radiology report using a same emphasis as the highlighting of the related anatomical feature.

2. The radiology viewer of claim 1 wherein:
the instructions readable and executable by the electronic processor to retrieve or generate a set of image tags and a set of report tags operate to generate sets of image tags identifying anatomical features in all radiology images stored in the radiology examinations data storage and to generate sets of report tags identifying clinical concepts in passages of all radiology reports stored in the radiology examinations data storage;
the instructions readable and executable by the electronic processor to receive a selection of an anatomical feature shown in the image window and to identify at least one related passage of the radiology report operates to identify all related passages in all radiology reports of all radiology examinations of the same patient in the radiology examinations data storage; and
the instructions readable and executable by the electronic processor to receive a selection of a passage of the radiology report shown in the report window and to identify at least one related anatomical feature of the at least one radiology image operates to identify all related anatomical features in all radiology images of all radiology examinations of the same patient in the radiology examinations data storage.

3. The radiology viewer of claim 1 wherein the instructions readable and executable by the electronic processor to generate the set of image tags perform operations including:
spatially registering the at least one radiology image with an anatomical atlas; and
generating the set of image tags by associating image features of the anatomical atlas with corresponding spatial regions of the spatially registered at least one radiology image.

4. The radiology viewer of claim 1 wherein the instructions readable and executable by the electronic processor to generate the set of report tags perform operations including:
identifying keywords in the radiology report with entries of the medical ontology; and
generating the set of report tags by associating passages of the radiology report containing the identified keywords with clinical concepts described in the corresponding entries of the medical ontology.

5. The radiology viewer of claim 1 wherein the instructions readable and executable by the electronic processor to generate the set of report tags perform operations including:
performing natural language processing on the radiology report to identify passages of the radiology report corresponding with entries of the medical ontology; and
generating the set of report tags by associating the identified passages of the radiology report with clinical concepts described in the corresponding entries of the medical ontology.

6. The radiology viewer of claim 7 wherein:
the receiving via the at least one user input device of the selection of the anatomical feature shown in the image window and the highlighting of the related passage of the radiology report in the report window further includes displaying a connecting arrow connecting the selected anatomical feature and the related passage of the radiology report; and
the receiving via the at least one user input device a selection of a passage of the radiology report shown in the report window and the highlighting of the related anatomical feature of the at least one radiology image in the image window includes displaying a connecting arrow connecting the selected passage of the radiology report and the related anatomical feature.

7. The radiology viewer of claim 1 wherein the instructions readable and executable by the electronic processor to display at least a portion of the at least one radiology image in an image window shown on the at least one display and to display at least a portion of the radiology report in a report window shown on the at least one display operate to display the image window and the report window simultaneously on the at least one display.

8. The radiology viewer of claim 1 wherein the electronic processor includes:
a server computer connected to read and execute the instructions to generate the set of image tags and the set of report tags and to further store the set of image tags and the set of report tags in the radiology examinations data storage; and
a viewer workstation operatively connected with the at least one display and the at least one user input device.

9. The radiology viewer of claim 8 wherein the viewer workstation is connected with the server computer and the radiology examinations data storage via the Internet.

10. A non-transitory storage medium storing instructions readable and executable by an electronic processor operatively connected with at least one display, at least one user input device, and a radiology examinations data storage to perform a radiology viewing method operating on at least one radiology image and a radiology report, the radiology viewing method comprising:
displaying at least a portion of the at least one radiology image in an image window shown on the at least one display;
displaying at least a portion of the radiology report in a report window shown on the at least one display; and
using a set of image tags identifying anatomical features in the at least one radiology image, a set of report tags identifying clinical concepts in passages of the radiology report, and an electronic medical ontology, at least one of:
(1) receiving via the at least one user input device a selection of an anatomical feature shown in the image window, identifying at least one related passage of the radiology report, and highlighting the at least one related passage of the radiology report in the report window; and
(2) receiving via the at least one user input device a selection of a passage of the radiology report shown in the report window, identifying at least one related anatomical feature of the at least one radiology image, and highlighting the at least one related anatomical feature of the at least one radiology image in the image window;
wherein the operation further identifies absence of any related passage in any radiology report of the radiology examinations of the same patient in the radiology examinations data storage, and the operation further identifies absence of any related anatomical feature in the radiology images of any radiology examination of the same patient in the radiology examinations data storage.

11. The non-transitory storage medium of claim 10 wherein the operation identifies related passages of the radiology reports of all radiology examinations of the same patient in the radiology examinations data storage, and the operation identifies related anatomical features of the radiology images of all radiology examinations of the same patient in the radiology examinations data storage.

12. (canceled)

13. The non-transitory storage medium of claim 10 wherein the radiology viewing method further comprises:
spatially registering the at least one radiology image with an anatomical atlas; and
generating the set of image tags by associating image features of the anatomical atlas with corresponding spatial regions of the spatially registered at least one radiology image.

14. The non-transitory storage medium of claim 10 wherein the radiology viewing method further comprises:
identifying passages in the radiology report with entries of the medical ontology; and
generating the set of report tags by associating the identified passages of the radiology report with clinical concepts described in the corresponding entries of the medical ontology.

15. The non-transitory storage medium of claim 10 wherein the displaying of at least a portion of the at least one radiology image in the image window and the displaying of at least a portion of the radiology report in the report window are performed concurrently such that the image window and the report window are shown simultaneously on the at least one display.

16. The non-transitory storage medium of claim 10 wherein:
operation includes highlighting the related passage of the radiology report in the report window using a highlighting color or pattern and further includes highlighting the selected anatomical feature in the image window using a same color or pattern as is used to highlight the related passage; and
operation includes highlighting the related anatomical feature of the at least one radiology image in the image window using a highlighting color or pattern and further includes highlighting the selected passage of the radiology report in the report window using the same color or pattern as is used to highlight the related anatomical feature.

17. A radiology viewing method operating on at least one radiology image and at least one radiology report, the radiology viewing method comprising:
displaying, on at least one display, an image window showing at least a portion of the at least one radiology image;
displaying, on the at least one display, a report window showing at least a portion of the at least one radiology report;
receiving via at least one user input device a selection of one of (i) an anatomical feature shown in the image window or (ii) a report passage shown in the report window;
identifying one of (i) at least one passage of the at least one radiology report corresponding to the selected anatomical feature or (ii) at least one anatomical feature of the at least one radiology image corresponding to the selected report passage, the identifying being performed by an electronic processor operating on a set of image tags identifying anatomical features in the at least one radiology image, a set of report tags identifying clinical concepts in passages of the at least one radiology report, and an electronic medical ontology; and
highlighting one of (i) the identified at least one passage of the radiology report in the report window or (ii) the identified at least one anatomical feature of the at least one radiology image in the image window.

18. The radiology viewing method of claim 17 wherein:
the receiving comprises receiving a selection of an anatomical feature shown in the image window;
the identifying comprises identifying at least one passage of the at least one radiology report corresponding to the selected anatomical feature; and
the highlighting comprises highlighting the identified at least one passage of the at least one radiology report in the report window.

19. The radiology viewing method of claim 18 wherein the highlighting further comprises highlighting the selected anatomical feature in the image window using a same highlighting as the highlighting of the identified at least one passage in the report window.

20. The radiology viewing method of claim 17 wherein:
the receiving comprises receiving a selection of a report passage shown in the report window;
the identifying comprises identifying at least one anatomical feature of the at least one radiology image corresponding to the selected report passage; and
the highlighting comprises highlighting the identified at least one anatomical feature of the at least one radiology image in the image window.

21. The radiology viewing method of claim 20 wherein the highlighting further comprises highlighting the selected report passage and highlighting any other passages of the same radiology report of passages of other radiology reports, where the same anatomical feature has been identified, in the report window using a same highlighting as the highlighting of the identified anatomical feature in the image window.

22. The radiology viewing method of claim 17 further comprising:
spatially registering the at least one radiology image with an anatomical atlas; and
generating the set of image tags by associating image features of the anatomical atlas with corresponding spatial regions of the spatially registered at least one radiology image.

23. The radiology viewing method of claim 17 further comprising:
identifying passages in the radiology report with entries of the medical ontology; and
generating the set of report tags by associating the identified passages of the radiology report with clinical concepts described in the corresponding entries of the medical ontology.
